(19) United States
(12) Patent Application Publication
(10) Pub. No.: US 2019/0175577 A1
(43) Pub. Date: Jun. 13, 2019

(54) METHOD FOR TREATING AMYOTROPHIC LATERAL SCLEROSIS

(71) Applicant: UNIVERSITY OF MIAMI, Miami, FL (US)

(72) Inventor: Michael Benatar, Miami, FL (US)

(21) Appl. No.: 16/213,179

(22) Filed: Dec. 7, 2018

Related U.S. Application Data
(60) Provisional application No. 62/596,406, filed on Dec. 8, 2017.

(51) Int. Cl. A61K 31/4545; A61P 25/00

(52) U.S. Cl. CPC A61K 31/4545; A61P 25/00

(57) ABSTRACT

The disclosure provides a method of treating amyotrophic lateral sclerosis (ALS) in a subject in need thereof, the method comprising administering to the subject a daily dose of Arimoclomol exceeding 300 mg per day.

### GRANT FUNDING DISCLOSURE

This invention was made with government support under grant number R01 FD003517 awarded by the U.S. Food and Drug Administration Orphan Products Development (OPD) Program. The government has certain rights in the invention.

### FIELD OF DISCLOSURE

The disclosure relates to materials and methods for treating amyotrophic lateral sclerosis.

### BACKGROUND

Amyotrophic lateral sclerosis (ALS) is characterized by the formation of cytosolic aggregates that contain specific misfolded proteins in selected neuronal and glia cells. Ince et al., Journal of Neuropathology and Experimental Neurology 1998; 57:895-904. There is mounting evidence that these aggregates play a pathogenic role in disease initiation and propagation. Bergh et al., Proceedings of the National Academy of Sciences of the United States of America 2015; 112:4489-4494; Bidhendi et al., The Journal of Clinical Investigation 2016; 126:2249-2253. The estimated prevalence rate in the U.S. was 4.7 cases per 100,000 people for 2012 and 5.0 per 100,000 for 2013. Mehta et al., Surveillance Summaries/Aug. 5, 2016/65(8); 1-12. Life expectancy following diagnosis is relatively short, with many patients succumbing to respiratory failure. There are few treatments for ALS currently available in the U.S. There remains a need in the art for new therapeutic regimens for treating ALS.

### SUMMARY

The disclosure provides a method of treating amyotrophic lateral sclerosis (ALS) in a subject in need thereof. The method comprises administering to the subject a daily dose of Arimoclomol exceeding 300 mg per day. In various aspects, the daily dose of Arimoclomol is about 600 mg, optionally administered as three separate administrations of 200 mg. The method optionally comprises administering the daily dose of Arimoclomol for twelve months or more. In various aspects, the subject comprises an CuZn-Superoxide Dismutase (SOD1) mutation (i.e., a patient with ALS in whom a pathogenic SOD1 mutation has been identified/demonstrated). In some embodiments, the method comprises, prior to administration of Arimoclomol, detecting a SOD1 mutation in the subject.

### BRIEF DESCRIPTION OF THE FIGURES

FIG. 1: Inclusion and exclusion criteria of phase II clinical trial described herein.

FIGS. 2a and 2b: Permanent assisted ventilation (PAV)- and tracheostomy-free survival. (a) All Arimoclomol- and placebo-treated participants. At 12 months, 34% of Arimoclomol-treated and <21% of placebo-treated participants were alive (and without PAV or tracheostomy). (b) The subgroup of A4V participants. At 12 months, 29% of Arimoclomol-treated and 15% of placebo-treated participants were alive (and without PAV or tracheostomy).

FIG. 3: Efficacy outcomes an analysis of the phase II clinical trial described herein.

### DETAILED DESCRIPTION

The disclosure provides a method of treating amyotrophic lateral sclerosis (ALS) in a subject in need thereof. The method comprises administering to the subject a daily dose of Arimoclomol exceeding 300 mg per day. Arimoclomol is a co-inducer of the heat-shock protein (HSP) response and promotes natural folding of nascent proteins and refolding of misfolded proteins. Hargitai et al., Biochemical and biophysical research communications 2003; 307:689-695; Kalmar et al., Experimental neurology 2002; 176:87-97; Kieran et al., Nature medicine 2004; 10:402-405; Paul et al., Molecular and cellular biochemistry 2014; 386:45-61. The structure of Arimoclomol is provided:

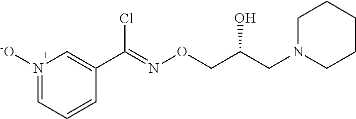

“Treating” ALS does not require 100% abolition of the disease or disease symptoms in the subject. Any relief or reduction in the severity of symptoms or features of the disease is contemplated. “Treating” ALS also refers to a delay in onset of symptoms or delay in progression of symptoms associated with the disease. The subject may not exhibit signs of ALS, although in many embodiments of the disclosure the subject exhibits early signs of the disease or displays symptoms of established or progressive disease. In various aspects, Arimoclomol is administered to the subject within 18 months of the initial onset of symptoms of ALS, e.g., within 12 months of the onset of symptoms, within nine months of the onset of symptoms, within six months of the onset of symptoms, or within three months of the onset of symptoms. The efficacy of the treatment may be characterized using any clinically acceptable method such as, for example, physical examination, laboratory testing, functional/behavioral testing (e.g., ALSFRS-R), imaging studies, electrophysiological studies, and the like. Clinical features associated with ALS include, e.g., lower motor neuron degeneration (e.g., weakness or wasting) in one or more of the bulbar, cervical, thoracic, and/or lumbosacral regions and upper motor neuron degeneration (e.g., increased tendon reflexes, spasticity, pseudo bulbar features, Hoffmann reflex, and/or extensor plantar response) in one or more of these regions. Additional clinical features include, but are not limited to, abnormal pulmonary function, speech problems, stumbling, abnormal swallowing, muscle cramps or stiffness, and weakness. Progression of the neuronal degeneration or muscle weakness is a hallmark of the disease. The disclosure contemplates any degree of alleviation of one or more symptoms of the disease or delay in the progression of any one or more disease symptoms (e.g., any improvement as measured by the ALSFRS-R or maintenance of an ALSFRS-R rating (signaling delayed disease progression)). In some embodiments, the subject is a mammal, such as a human.

The method of the disclosure comprises administering Arimoclomol to the subject in a daily dose exceeding 300 mg per day (e.g., the total amount of Arimoclomol administered over the course of day is more than 300 mg). The amount administered is sufficient to achieve a desired biologically effect in a clinically relevant timeframe while minimizing unwanted side effects. In various aspects, the daily dose is about 400 mg or more, about 500 mg or more, about 600 mg or more, about 700 mg or more, about 800 mg or more, or about 900 mg or more. For example, in some embodiments, the daily dose of Arimoclomol is about 400 mg to 1200 mg, about 400 mg to about 1000 mg, about 400 mg to about 800 mg, about 400 mg to about 600 mg, about 600 mg to about 1200 mg, about 600 mg to about 1000 mg, about 800 mg to about 1200 mg, or about 800 mg to about 1000 mg. The daily dose is optionally administered in multiple administrations throughout the day (i.e., the total daily dose is provided as two or more divided (or sub-) doses). For example, when the daily dose is about 600 mg, the daily dose may be administered as two administrations of 300 mg or three administrations of 200 mg. The administration regimen described herein provides a higher dose of Arimoclomol than previously contemplated and is well tolerated in human subjects, as described below. Further, the administration regimen described herein mediated clinically meaningful improvement in ALS patient health, especially in subjects comprising a SOD1 mutation (A4V).

Treatment of chronic diseases such as ALS generally entails repeated administration of a therapeutic for an extended period of time, such as daily administrations for one or more months (e.g., two, three, four, five, six, seven, eight, nine, ten, eleven, or twelve months). The therapeutic period may comprise a year (52 weeks or 12 months) or more. The Arimoclomol may be administered to the subject using any suitable route of administration, e.g., orally or parenterally.

While not wishing to be bound to any particular theory, Arimoclomol's mechanism of action is believed to be relevant to all forms of ALS in which aberrant proteostasis plays an essential role in disease pathophysiology. In one aspect, however, the subject comprises a mutation in the gene encoding CuZn-Superoxide Dismutase (SOD1). Mutation results in the SOD1 protein being more prone to aggregation, resulting in the deposition of cellular inclusions that contain misfolded SOD1 aggregates. Andersen et al., Nature reviews Neurology 2011; 7:603-615. Over 100 different mutations in SOD1 have been linked to inherited ALS, many of which result in a single amino acid substitution in the protein. In some embodiments, the SOD1 mutation is A4V (i.e., a substitution of valine for alanine at position 4). SOD1 mutations are further described in, e.g., Rosen et al., Hum. Mol. Genet. 3, 981-987 (1994); Rosen et al., Nature 362, 59-62 (1993).

In various aspects, the method comprises, prior to administration of Arimoclomol, detecting a SOD1 mutation in the subject. Methods of screening for mutations, including SOD1 mutations (e.g., at chromosome 21q22.1), are well known in the art. Suitable methods include, but are not limited to, genetic sequencing. See, e.g., Hou et al., Scientific Reports, 2016, 6:32478; Vajda et al., Neurology 2017, 88:1-9, all of which are incorporated by reference in their entirety and specifically with respect to description of methods of detecting SOD1 mutations.

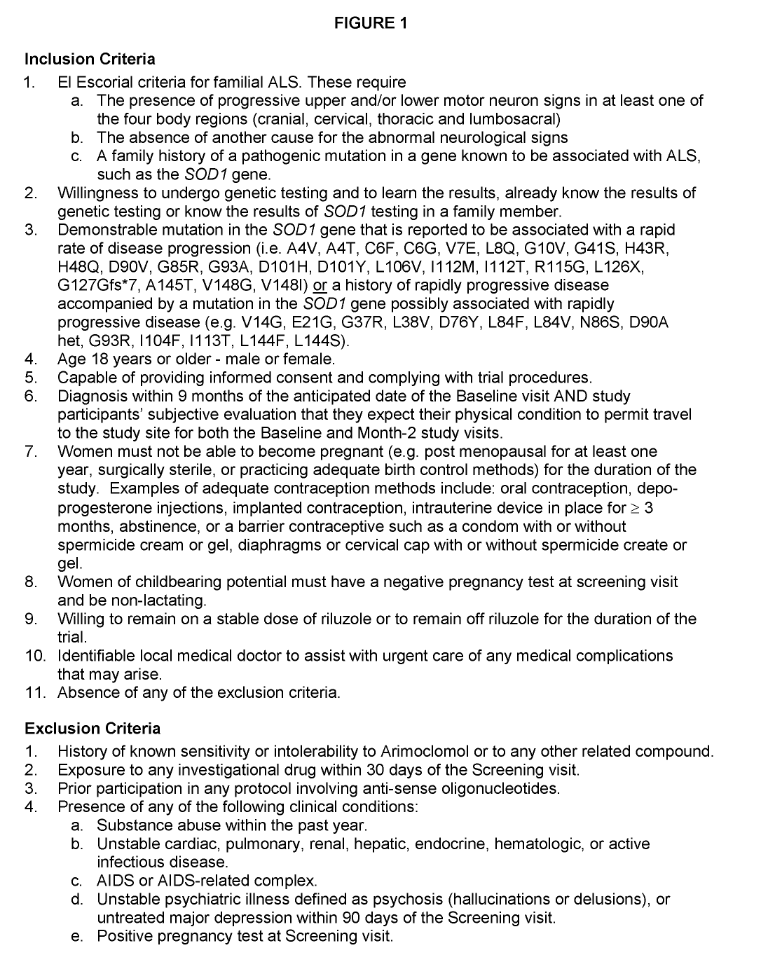

Provided herein are the details of a completed, randomized, double-blind placebo controlled phase II trial. The trial was conducted at two sites and three academic medical centers in the United States. An independent medical monitor completed regular review of laboratory reports and adverse events (AEs), as well as real-time review of serious adverse events (SAEs). Eligibility criteria (FIG. 1) aimed to enroll, from across the U.S. and Canada, a population relatively early in the course of ALS caused by SOD1 mutations associated with rapidly progressive disease.

Randomization (1:1 to Arimoclomol or matching placebo) was stratified by Riluzole use and in block size of two within each stratum, and implemented using a central web-based electronic data capture (EDC) system managed by the Neurological Clinical Research Institute Data Management Center at MGH. The randomization schedule, generated by a study statistician, was provided to each research pharmacy. At randomization, the EDC assigned a subject identification number, which the site-coordinator submitted to the research pharmacy for drug dispensing. Encapsulated placebo was matched in color and appearance to active drug. Except for the research pharmacists, pharmacy monitors, and study statisticians, all other personnel and study participants were blinded to treatment assignments.

The first participants were randomized and administered 100 mg of Arimoclomol three times daily. After enrollment of the first 16 participants (of whom 8 were on Arimoclomol, including 1 subject subsequently excluded per protocol), the dosage was increased to 200 mg administered three times daily for all active participants (which for the Arimoclomol group included three subjects initially started at 100 mg administered three times daily), and all newly enrolled participants received 200 mg three times a day. All data were analyzed following the intent-to-treat principle.

Assessments were performed in person only at Baseline and Month-2, with remote assessments planned for all other visits. Neurological examination, motor unit number estimation (MUNE), and slow vital capacity (SVC) could only be performed in person. Other assessments, conducted at all visits (in person and remotely), included: vital signs; blood and urine for safety labs; ALSFRS-R (revised ALS Functional Rating Scale), which has been validated for telephone administration; and the forced expiratory volume in 6-seconds (FEV6). Miano et al., Amyotrophic Lateral Sclerosis And Other Motor Neuron Disorders: Official Publication Of The World Federation Of Neurology, Research Group On Motor Neuron Diseases 2004; 5:235-239; Vandevoorde et al., Chest 2005; 127:1560-1564. In addition, AEs, concomitant medications, study drug dose management, use of ventilatory support, and key study events were recorded at all visits.

Operationalization of remote assessments posed logistical challenges that required the introduction of two innovative features: (1) the collection of vital signs and blood/urine for safety labs in participants' homes, this was accomplished using the services of a mobile medical provider; and (2) the use of FEV6 as the principal measure of respiratory function. The key advantage of FEV6 over SVC is that FEV6 may be self-administered by the participant using a low-cost portable device (e.g., Piko-6, nSpireHealth, Inc.). The device digitally displayed the FEV6 value, which the participant then reported to the study coordinator. Use of the FEV6 allowed for remote assessment of respiratory function, which otherwise would not have been available for collection in this trial. Reproducibility and normative data for FEV6 are established. Hankinson et al., Chest 2003; 124:1805-1811.

The primary endpoint of safety and tolerability was based on the frequency of AEs, abnormal vital signs, and abnormal laboratory studies above or below pre-defined alert levels. AEs and SAEs were categorized according to the Common Terminology Criteria for Adverse Events (CTCAE) and rated for severity and relatedness to study drug. In summarizing AEs: (a) if a participant experienced multiple occurrences of the same event, only the occurrence with the worst severity (or highest degree of relatedness to study drug) was counted; and (b) CTCAE events were further classified into subgroups (“AE type”), e.g., pneumonia and bronchitis were both classified as upper/lower respiratory infection.

For efficacy, the principal outcome measure was PAV- and tracheostomy-free survival time, calculated from baseline to PAV (defined as first of seven consecutive days when PAV was used >22 hours/day) or tracheostomy, date of death (if no PAV or tracheostomy), or date of last available follow-up/study contact (if still PAV- and tracheostomy-free by then). Participants not reaching survival endpoints were censored. Secondary efficacy measures included ALSFRS-R rate of decline (points/month); FEV6 rate of decline (% predicted/month); and joint rank scores of the Combined Assessment of Function and Survival (CAFS), which considers both ALSFRS-R rate of decline and survival.

All 36 eligible participants who completed at least one follow-up visit were included in the intent-to-treat analysis. The only pre-defined subgroup was the A4V SOD1 mutation carriers. PAV- and tracheostomy-free survival was first summarized by Kaplan-Meier survival estimates and compared between treatment groups by Wilcoxon and log-rank tests, then analyzed using a proportional hazards model with Riluzole use and baseline ALSFRS-R as pre-specified covariates. ALSFRS-R and FEV6 rates of decline were compared between groups by mixed model analysis with a random intercept and slope, and the outcome measure at each visit as dependent variable. The independent variables were time and time-treatment interaction, with the test of treatment effect based on the time-treatment interaction. In secondary analyses, a quadratic term for time was included. In addition, two analyses that combine survival and ALSFRS-R rate of decline were performed. The CAFS joint rank scores were compiled for each participant and compared between groups by two-sample t-test. Berry et al., Amyotrophic lateral sclerosis & frontotemporal degeneration 2013; 14:162-168. Treatment effect on the ALSFRS-R rate of decline, as well as any treatment effect on survival that is mediated through the ALSFRS-R, were estimated by the Vonesh shared parameter model. Vonesh et al., Statistics in medicine 2006; 25:143-163. Numerical time, based on actual number of days between baseline and each follow-up visit, was used in the survival, mixed model, and Vonesh analyses. For CAFS, survival times and ALSFRS-R rates of decline were obtained using numerical time, but the participant-to-participant comparisons made at nominal time points. Baseline covariate adjustment for potential imbalance between groups was considered. All analyses were performed using SAS 9.3.

The results of the study are as follows. Adverse events (AEs) that occurred in ≥10% (N≥4) of participants were generally mild, occurring with similar frequency in the two arms and largely considered unrelated to study drug. Twenty-two serious adverse events (SAEs) were reported (15 in placebo and 7 in Arimoclomol group), none of which were considered related to study drug. Abnormal vital signs or laboratory values were infrequent and occurred with comparable frequency in the two groups. A single participant stopped Arimoclomol because of a skin rash.

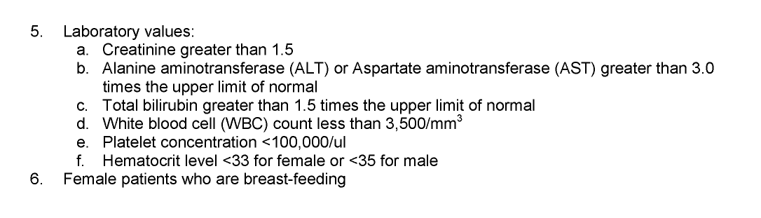

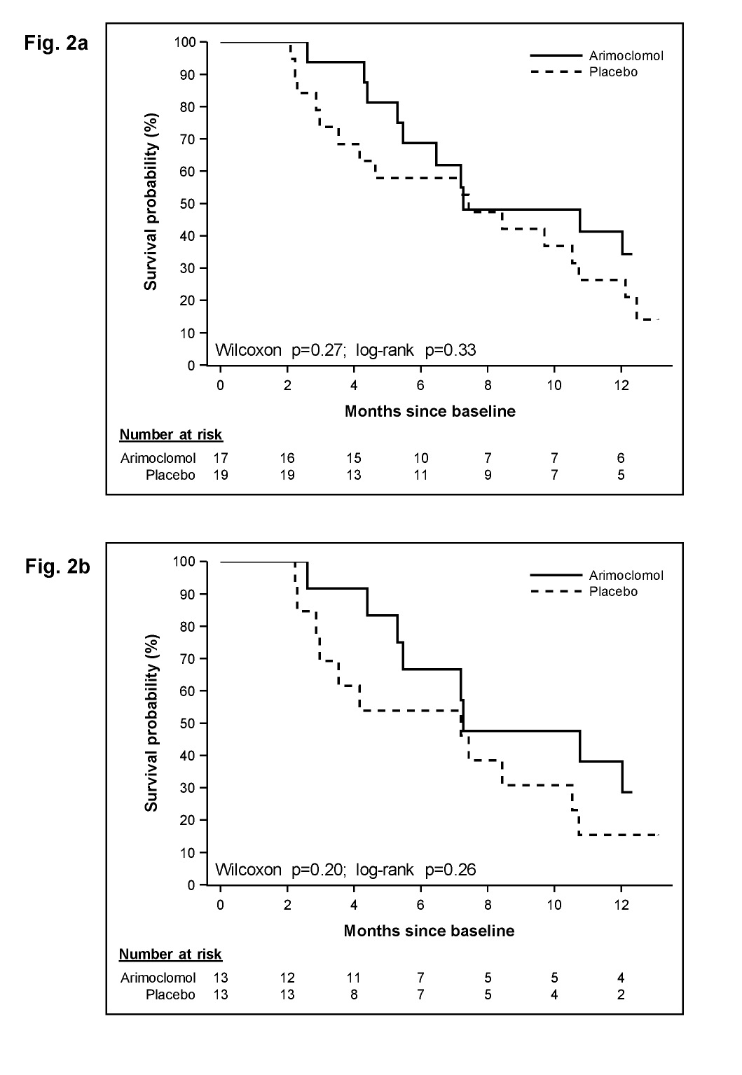

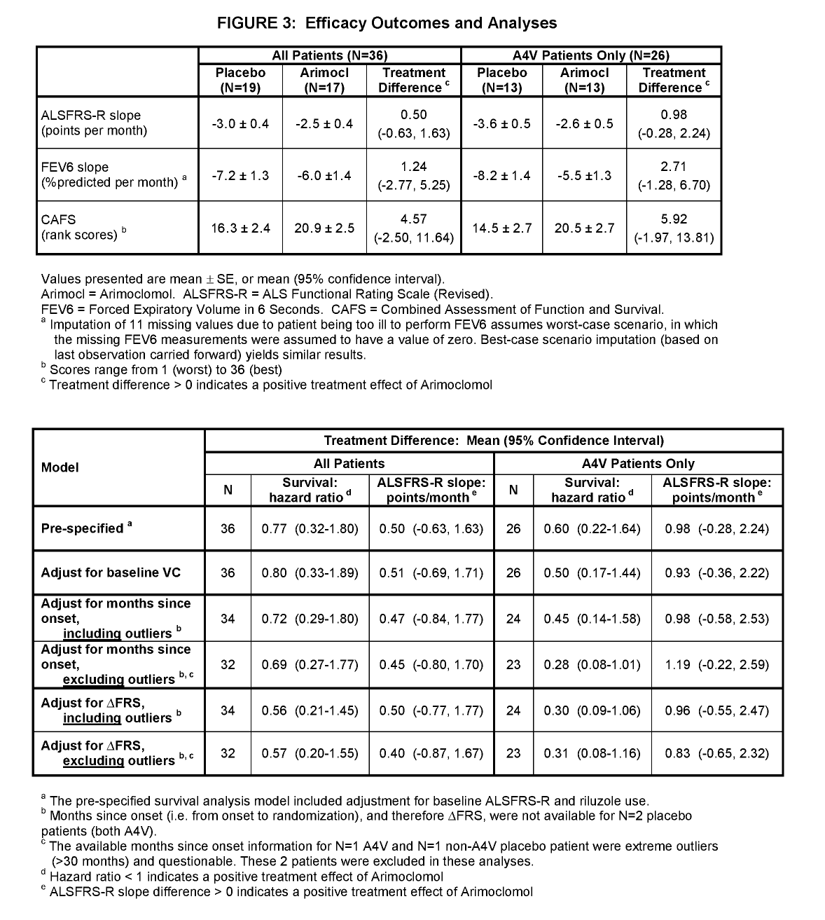

All point estimates favored Arimoclomol treatment but with confidence intervals spanning unity. Kaplan-Meier plot shows a separation in survival curves, though the curves slightly crossed around 7.5 months (Wilcoxon test p=0.27, log-rank test p=0.33) (FIG. 2a). Similar results are observed in the A4V subgroup (N=13/group) (FIG. 2b). Survival estimates from the Cox proportional hazards model also favored Arimoclomol compared to control, with an unadjusted hazard ratio (HR) of 0.67 (95% CI: 0.29-1.48, p=0.33). Adjusting for baseline ALSFRS-R and Riluzole, HR=0.77 (95% CI: 0.32-1.80, p=0.55). Adjustment for the baseline imbalance in respiratory function did not affect results (FIG. 3).

Among placebo-treated participants ALSFRS-R declined by an average (±SE) of 3.0±0.4 points/month, compared to 2.5±0.4 points/month in the Arimoclomol-treated group, a treatment difference of 0.5 points/month (95% CI: −0.63 to 1.63, p=0.37). In the A4V subgroup, ALSFRS-R declined even faster—by an average of 3.6±0.5 points/month in the placebo group but only 2.6±0.5 points/month in the Arimoclomol group, a treatment difference of 0.98 points/month (95% CI: −0.28 to 2.24, p=0.12). Notably, the magnitude of this difference is comparable to the average ALSFRS-R rate of decline in the untreated general ALS population (1.02±2.3 point/month). Moreover, the treatment difference of 0.5-1.0 points/month in ALSFRS-R rate of decline, though not statistically significant, translates into a clinically meaningful difference of 6-12 points over a one-year period. Similar results were observed for the FEV6% predicted rate of decline (FIG. 3). Moreover, while adjustment for baseline ALSFRS-R or respiratory function variably increased or decreased the magnitude of treatment effect, overall results were unchanged (FIG. 3).

For the CAFS, which weights mortality as the more clinically important outcome and whose rank scores range from 1 (worst) to 36 (best), the average (±SE) score in the Arimoclomol group was 20.9±2.5 compared to 16.3±2.4 in the placebo group, with a potential treatment benefit of 4.57 points (95% CI: −2.50 to 11.64) (FIG. 3). Moreover, in participant-to-participant comparison between the two groups, the Arimoclomol group had a clinically significant win ratio of 1.6924. The Vonesh model, on the other hand, yielded a treatment difference of 0.77±0.54 points/month (95% CI: −0.33 to 1.86, p=0.16) in ALSFRS-R rate of decline, whereas the direct effect of treatment on survival was not significant (p=0.62). This would indicate that the possible survival benefit of Arimoclomol was mediated through a slowed decline of the ALSFRS-R rather than through an independent effect on survival.

Arimoclomol was safe and well tolerated at a dose of 600 mg per day, administered as three 200 mg administrations. The consistency of results across all pre-specified efficacy measures establishes a therapeutic benefit of Arimoclomol in the overall study population and especially in the A4V subgroup. The trial is significant in that it represents the first ALS trial initiated in a genetically and phenotypically homogeneous population. Although logistically challenging, this approach is both feasible and highly relevant to future trials, as the importance of targeting drugs with particular mechanisms of action to patient subpopulations most likely to benefit is realized. Additionally, this trial pioneered an approach in which safety and efficacy assessments relied heavily on remote assessments. The study introduced FEV6 to the ALS field and demonstrated its usefulness in reliably quantifying respiratory function; FEV6 may be a useful alternative to vital capacity when the latter cannot readily be obtained. Further, this trial provides the first prospectively acquired natural history data that informs survival in the mutant SOD1-population.

1. A method of treating amyotrophic lateral sclerosis (ALS) in a subject in need thereof, the method comprising administering to the subject a daily dose of Arimoclomol exceeding 300 mg per day.

2. The method of claim 1, wherein the method comprises administering the daily dose of Arimoclomol for twelve months or more.

3. The method of claim 1, wherein the daily dose of Arimoclomol is about 600 mg.

4. The method of claim 1, wherein the daily dose of Arimoclomol is administered as three separate administrations of 200 mg.

5. The method of claim 1, wherein the subject comprises an CuZn-Superoxide Dismutase (SOD1) mutation.

6. The method of claim 1, wherein the method comprises, prior to administration of Arimoclomol, detecting a SOD1 mutation in the subject.

7. The method of claim 2, wherein the daily dose of Arimoclomol is about 600 mg.

8. The method of claim 2, wherein the daily dose of Arimoclomol is administered as three separate administrations of 200 mg.

9. The method of claim 3, wherein the daily dose of Arimoclomol is administered as three separate administrations of 200 mg.

10. The method of claim 7, wherein the daily dose of Arimoclomol is administered as three separate administrations of 200 mg.

11. The method of claim 2, wherein the subject comprises an CuZn-Superoxide Dismutase (SOD1) mutation.

12. The method of claim 3, wherein the subject comprises an CuZn-Superoxide Dismutase (SOD1) mutation.

13. The method of claim 4, wherein the subject comprises an CuZn-Superoxide Dismutase (SOD1) mutation.

14. The method of claim 9, wherein the subject comprises an CuZn-Superoxide Dismutase (SOD1) mutation.

15. The method of claim 2, wherein the method comprises, prior to administration of Arimoclomol, detecting a SOD1 mutation in the subject.

16. The method of claim 3, wherein the method comprises, prior to administration of Arimoclomol, detecting a SOD1 mutation in the subject.

17. The method of claim 4, wherein the method comprises, prior to administration of Arimoclomol, detecting a SOD1 mutation in the subject.

18. The method of claim 7, wherein the method comprises, prior to administration of Arimoclomol, detecting a SOD1 mutation in the subject.

19. The method of claim 9, wherein the method comprises, prior to administration of Arimoclomol, detecting a SOD1 mutation in the subject.

20. The method of claim 10, wherein the method comprises, prior to administration of Arimoclomol, detecting a SOD1 mutation in the subject.
